(19) United States
(12) Patent Application Publication
(10) Pub. No.: US 2005/0143346 A1
(43) Pub. Date: Jun. 30, 2005

(54) Composition for treating cancer containing n,n-dimethylphytosphingosine

(76) Inventors: Jin-Hee Choi, Seoul (KR); Chang-Seo Park, Gwancheon-city (KR); Jin-Wook Kim, Yongin-city (KR); Chang-Yeol Park, Yong-in city (KR); You-A Hwang, Gwangjoo-gun (KR); Eun-Ju Kim, Yong-in city (KR); Ui-Chan Koh, Seoul (KR)

Correspondence Address: MERCHANT & GOULD PC, P.O. BOX 2903, MINNEAPOLIS, MN, 55402-0903, US

(21) Appl. No.: 10/512,126

(22) PCT Filed: May 1, 2003
(86) PCT No.: PCT/KR03/00882

(30) Foreign Application Priority Data
May 2, 2002 (KR) 1020020024245
Jan. 28, 2003 (KR) 1020030005603

(51) Int. Cl. C07F 9/02; A61K 31/685

(52) U.S. Cl. 514/78; 554/78

(57) ABSTRACT

A composition and a kit for treating cancer comprising N,N-dimethylphytosphingosine. The composition represses the activity of sphingosine kinase, and therefore, intercepts various mechanisms which sphingosine kinase induces. For example, the composition blocks the phosphorylation of ceramide and sphingosine, thereby maintaining high concentration of ceramide and sphingosine. The ceramide and sphingosine induce apoptosis in cancer cells. Therefore, the composition according to the present invention induces apoptosis in cancer cells and accordingly kills the cancer cells.

### FIELD OF THE INVENTION

The present invention relates to a composition comprising dimethylphytosphingosine, and more specifically to a composition which has an inhibitory activity of sphingosine kinase, an inhibitory activity of protein kinase C (PKC), an apoptosis inducing activity, a treating activity of hyperplastic disease, an anti-cancer activity, an anti-inflammatory activity and an anti-bacterial activity.

### BACKGROUND OF THE INVENTION

A major obstinate disease, cancer, is the current leading cause of death and its incident rate is steadily increasing. Surgery, radiotherapy and chemotherapy are the most common treatment for cancer, but currently only 50% of treated patients are completely recovered. If cancer is detected at early stage, it may be completely cured by surgical operation or radiotherapy. However, for progressed cancer, chemotherapy is used alternatively. The remedial value of chemotherapy is relatively low due to its limited dosage and treatment period. Chemotherapy is frequently accompanied by side effects and drug resistance.

Recently, as a consequence of active researches and studies, the targets for anti-cancer drug development have been diversified, and it led to development of very effective drugs with little side effect. In addition, many ongoing researches are related to combination therapies that improve drug efficacy.

The ultimate goal of anti-cancer drug development are to develop drugs selectively effective on cancer cells and to develop drugs not causing any drug resistance. A mechanisms of most existing anti-cancer drug is to kill the cancer cells by increasing its ceramide pool. The mechanism is interpreted as follows: Foreign stimuli such as anti-cancer drug and radiation cause decomposition of sphingomyelin and augmentation of ceramide level which induce apoptosis of cells. However, if sphingosine converts into phosphorylated sphingosine by sphingosine kinase, cancer cells cannot by killed by apoptosis, rather be proliferated by its growth inducement. Meanwhile, recent studies have been reported that large amount of glycosylceramide produced by glycosylceramide synthetase is related to drug resistance.

Consequently, the action mechanism of anti-cancer drugs is to induce a change of the level of ceramide in cancer cells. Particularly, the substance which increases the level of ceramide or inhibits degradation thereof has been expected as an excellent candidate of anti-cancer drugs, since the fact that for cancer cell the level of ceramide is lower than that of normal cell has been reported. Accordingly, various targets can be chosen from the metabolic pathway of ceramide. An approaching method is to affect ceramide synthetase to increase new synthesis of ceramide. The substances which have been ever known include Paclitaxel, Etoposide, Anthracyclines, Vinca alkaloids, C6-Ceramide, TNF-α, SDZ PSC 833 and the like. Further, another goal of development of anti-cancer drugs is to increase the level of ceramide by affecting sphingomyelinase to promote degradation of sphingomyelin. Such effects can be achieved by using irradiation, CD-95, Anthracyclines, TNF-α, Fas ligand, Ara-C and the like. It is also important to increase the level of ceramide in cells, but to inhibit degradation of produced ceramide is also a good target of anti-cancer drug development. Among them, an approach to inhibit synthesis of glycosylceramide which induces drug resistance can be used effectively to enhance the effect of anti-cancer drugs. Up to date, Tamoxifen, Toremifene, Mifepristone, Cyclosporin A, Keroconazole, Verapamil, PPMP and the like were known as substances which have such effect. Such substances can be used together with substances which promote the synthesis of ceramide rather than its single use, to enhance the effect of anti-cancer drugs. Another approach which inhibits degradation of ceramide is to inhibit the activity of sphingosine kinase or to activate sphingosine phosphatase which converts phosphorylated sphingosine to sphingosine by removing the phosphate group from phosphorylated sphingosine. A representative example of substances which inhibit activity of sphingosine kinase is dimethylsphingosine. Also, as an inhibitor of protein kinase is known to enhance death effect of cancer cells when it is used together with anti-cancer drugs, recently various researches are commenced to maximize anti-cancer effect by using a combination of anti-cancer drugs and substances which have such effect.

Synergistic effect can be achieved by using a combination of anti-cancer drugs and substances which inhibit degradation of ceramide in amount which has no effect on the cells, and also risk of side effect can be reduced.

Use of a combination of ceramide and taxol has been reported to increase apoptosis of Head and Neck cancer cell. Also, it has been known that ceramide level of rectal cancer cell is at most 50% of that of normal cell, and that strong inhibitor of ceramidase can induce apoptosis. Particularly, an inhibitor of ceramidase can be a good target of anti-cancer drugs, since it has no selectivity on cancer cells. Further, a clinical environment showed that use of a Safingol(L-threo-dihydrosphingosine) as an inhibitor of protein kinase together with a kind of anti-cancer drug, Doxorubicin increases its anti-cancer effect. Meanwhile, sphingosine kinase is believed to have a property of oncogene, since the speed of cell division was raised and the transformed aspect was represented when sphingosine kinase was expressed in NIH 3T3 fibroblast. By the way, an inhibitor of sphingosine kinase can be used as anti-cancer drugs, since an increase of expression of sphingosine kinase has been reported to prevent apoptosis.

Various prior researches have been reported. These researches showed that a combination of Safingol(L-threo-dihydrosphingosine), a competitive inhibitor of protein kinase and Doxorubicin or Mitomycin C increased the effect of cancer cell death, and also induced cytotoxicity against anti-cancer drug resistant cell lines (U.S. Pat. No. 6,444,638, U.S. Pat. No. 5,821,072 and the like). Further, U.S. Pat. No. 6,368,831 and the like showed that an inhibitor of ceramide degradation had a good effect on treating hyperplastic disease. For above mentioned patents, a combination of various anti-cancer drugs and dimethylsphingosine as an inhibitor of ceramide degradation was used.

Recently, M. D. Anderson cancer center reported that dimethylsphingosine has an effect on a drug resistant acute leukemia.

Sphingosine or Phytosphingosine and its derivatives have been reported to have various functions including above mentioned functions. Phosporylated sphingosine is a second messenger on cell proliferation involved with PDGF (platelet derived growth factor). Also, it was known that they are contained in platelets at high level, activates platelets, and released from platelets to play a role in pathophysiological functions such as hemostasis, thrombosis, wound healing and the like. Further, they functioned as a first messenger modulating mobility of cell. Sphingosine is a PKC inhibitor produced by ceramidase, and plays a role of inducing apoptosis of cancer cells. Dimethylsphingosine(N,N-dimethylsphingosine) among methylated sphingosines is referred to metabolically stable sphingosine, and also its functions are similar with those of sphingosine. However, dimethylsphingosine is stronger PKC inhibitor than sphingosine, and is an apoptosis inducing substance which suppress growth of epidermis cancer cells, leukemia cells as well as various cancer cells. And they also activate platelets and inhibit release of phosphorylated sphingosine. Trimethylsphingosine(N,N,N-trimethylsphingosine) has a strong PKC inhibiting effect similar with dimethylsphingosine, and is a improved substance in views of cytotoxicity and solubility in water. But, it has no apoptosis inducing function, and has little function which inhibits sphingosine phosphatase compared to dimethylsphingosine. However, it has been reported that its anti-inflammatory effect is excellent (Igarashi, Y. 1997 J. Biochem. 122, 1080-1087). Mostly, studies regarding metabolism of phytosphingosine were performed in yeast, but substantially it was found that phytosphingosine was presented in epidermis of human as well as yeast, and that it had an inhibitory effect against PKC and PLD (Phospholipase D), and ultraviolet-induced inflammation in vivo. Also, it was shown that phytosphingosine had an excellent effect which suppress growth of propionibacterium acnes and staphylococcus aureus compared to erythromycin (KR Patent Application No. 2001-15700, Park Changseo et al.; KR Patent Application No. 2000-74074, Kim Jinwook et al.; U.S. Ser. No. 09/691,446 Park Changseo et al.).

Such physiologically active substance, phytosphingosine has a notable function, but is so difficult to use for the reason of economics. Because it is very expensive for producing by pure synthetic method. Further, in many cases since stereochemical structures of synthetic sphingolipid are different from those of sphingolipid presented in human, and also for the method with extraction, the origin of synthetic sphingolipid is controversial, its use has been limited. Under these circumstances, the present inventors developed a microbiological process for preparing phytosphingosine in large amount under optimal fermentation conditions (KR Patent No. 221357; U.S. Pat. No. 5,958,742; FR Patent No. 2871502), using novel yeast cell which was isolated form parent cell (NRRL Y-1031 (F-60-10)) by means of spore isolation (KR Patent No. 188857; U.S. Pat. No. 6,194,196). It was found that stereochemical structures of phytosphingosine obtained by above mentioned method are identical to those of sphingolipid presented in human, thus its industrial applicability has been increased. Accordingly, various derivatives, which have excellent bioactivity, were developed by using mass producible phytosphingosine as a base compound.

### DETAILED DESCRIPTION OF THE INVENTION

Accordingly, the present invention has been made to solve the above-mentioned problems occurring in the prior art, and an object of the present invention is to treat or prevent cancer by maintaining a high level of ceramide and sphingosine in the targeted cells which induce apoptosis by inhibiting the activity of sphingosine kinase. Further, another object is to treat or prevent a hyperplastic disease such as cancer and psoriasis by inhibiting the activity of protein kinase and the activity of sphingosine kinase which promotes cell proliferation. Also, further another object is to provide a composition bearing excellent apoptosis inducing activity as such. Further, another object is to provide a composition bearing excellent antibacterial activity, anti-inflammatory activity and the like.

To achieve the above mentioned objects, an anti-cancer composition according to the present invention is characterized by comprising N,N-dimethylphytosphingosine as an active ingredient.

For the above mentioned anti-cancer compositions, the compositions are characterized by further comprising at least one phytosphingosine derivatives selected from the group consisting of phytosphingosine, acetylated phytosphingosines and ethylated phytosphingosines.

The most preferable, weight ratio of the N,N-dimethylphytosphingosine to the phytosphingosine derivatives is 1:1.

Further, the present invention provides a kit for treating cancer, and the kit is characterized by comprising the above mentioned anti-cancer composition.

Also, the above mentioned kit is characterized by comprising the composition as an adjuvants of other anti-cancer drugs.

Further, the present invention provides sphingosine kinase inhibiting composition, apoptosis inducing composition, inhibitory composition of protein kinase C, anti-inflammatory composition, or a composition for treating hyperplastic disease and antibacterial composition, which has N,N-dimethylphytosphingosine as an active ingredient.

For the above mentioned composition for treating hyperplastic disease, the hyperplastic disease is psoriasis.

A chemical structure of dimethylphytosphingosine of which the composition of the present invention comprises is as follows:

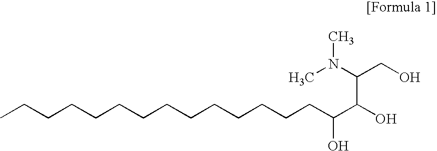

N,N-dimethylphytosphingosine of formula 1 can be produced with the process of reacting phytosphingosine of the formula 2 with the formaldehyde in a solvent in the presence of a reducing agent via the compound of the formula 3 as an intermediate:

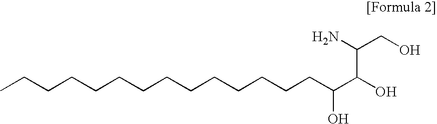

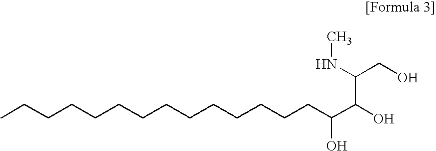

### BRIEF DESCRIPTION OF THE DRAWINGS

The above and other objects, features and advantages of the present invention will be more apparent from the following detailed description taken in conjunction with the accompanying drawings, in which:

FIG. 1 illustrates a 1H NMR spectrum of N,N-dimethylphytosphingosine(DMPS) according to the present invention;

FIG. 2 illustrates a MALDI-MASS spectrum of N,N-dimethylphytosphingosine according to the present invention;

FIG. 3 illustrates a graph representing the inhibitory effect on sphingosine kinase of N,N-dimethylphytosphingosine according to the present invention;

FIG. 4 illustrates a graph representing the apoptosis inducing effect of N,N-dimethylphytosphingosine according to the present invention on HL60 cell line;

FIG. 5 illustrates a graph representing the apoptosis inducing effect of N,N-dimethylphytosphingosine according to the present invention on HaCaT cell line;

FIG. 6 Illustrates a graph representing the apoptosis inducing effect of N,N-dimethylphytosphingosine according to the present invention on LLC-PK1;

FIG. 7 illustrates a graph representing the apoptosis inducing effect of N,N-dimethylphytosphingosine according to the present invention on B104 cell line;

FIG. 8 illustrates a graph representing the apoptosis inducing effect of N,N-dimethylphytosphingosine according to the present invention on MDA-MB-231 cell line;

FIG. 9 illustrates a result of electrophoresis, which represents the DNA fragmentation effect of N,N-dimethylphytosphingosine according to the present invention;

FIG. 10 illustrates a graph representing the increasing effect the binding capacity of anti-histone antibiodes by N,N-dimethylphytosphingosine according to the present invention;

FIG. 11 illustrates a graph representing the inhibitory effect of PKC by N,N-dimethylphytosphingosine according to the present invention; and

FIG. 12 illustrates a graph representing the antibacterial activity of N,N-dimethylphytosphingosine according to the present invention.

### DETAILED DESCRIPTION OF THE PREFERRED EMBODIMENTS

Hereinafter, preferred embodiments of the present invention will be described.

The process for preparation can be described more specifically as follows: The process used reductive methylation based on amine methylation of protein. At this time, hydride, preferably sodium borohydride, can be used to increase the reactivity of amine, and the amount is 8.0 to 10.0-fold (molar basis) based on the amount of compound of formula 2. A 1:1 mixture of borate buffer and methanol was used as a solvent. The reaction was performed at ambient temperature for 72 hrs by adding a specific amount of aqueous 37% formaldehyde solution several times at intervals.

In the present invention, various oxidizing agents which are conventionally known in the art and have no effect on the reaction can be used. Further, the compound of formula 1 produced as described above could be extracted with an organic solvent such as chloroform, a mixture of chloroform and methanol and purified by silica gel adsorption chromatography.

Dimethylphytosphingosine contained in the composition of the present invention has superior inhibitory effect of sphingosine kinase to dimethylsphingosine. Particularly, the present inventions have found that dimethylphytosphingosine induced strong apoptosis exclusively on several cancer cell lines, since it has a superior apoptosis inducing effect, an inhibitory effect of protein kinase C and the like.

Dimethylphytosphingosine can be used as a medicament as such or in the form of pharmaceutically acceptable salts. Examples of these salts include, but are not limited to, hydrochloric acid, sulphuric acid, nitric acid, phosphoric acid, hydrofluoric acid, hydrobromic acid, formic acid, acetic acid, tartaric acid, lactic acid, citric acid, fumaric acid, maleic acid, succinic acid, methanesulfonic acid, benzenesulfonic acid, toluenesulfonic acid, naphthalenesulfonic acid and the like.

The composition of the present invention can be formulated in the form of a pharmaceutical composition of anti-cancer drugs or anti-cancer drug enhancers comprising dimethylphytosphingosine. In this event, the composition comprises, if necessary, any adjuvants which have no adverse effect on the active ingredient, for example carrier or other additives such as stabilizer, relaxant, emulsifier and the like.

Also, the composition comprising dimethylphytosphingosine of the present invention can be administrated orally or parenterally. Further, the composition may be in a form suitable for a administration mode, for example injections, powders, granules, tablets and the like.

Dimethylphytosphingosine can be used exclusively or together with other phytosphingosine derivatives, in the latter case, most preferably content ratio of two components is 1:1. That is, a combination of dimethylphytosphingosine and other phytosphingosine derivatives comprises 50% of dimethylphytosphingosine and 50% of phytosphingosine derivatives. For pharmaceutical dosage forms, the content of the combination varies with the formulation types according to a conventional procedure. Preferable dosage amount of dimethylphytosphingosine according to the present invention is 0.001 to 1000 mg/kg·day.

The composition of the present invention can be administrated exclusively or together with other anti-cancer drugs as equivalents or adjuvants thereof.

Meanwhile, the composition of the present invention can be a cosmetic composition, examples of the cosmetic composition form include, but are not limited to skin softener, astringent, skin lotion, essence, lotion, nutrition cream, gel, pack, cleansing cream, cleansing foam, cleansing water and so on.

### EXAMPLES

The invention will be illustrated more specifically by the following non-limiting examples:

### Example 1

### Preparation of N,N-dimethylphytosphingosine

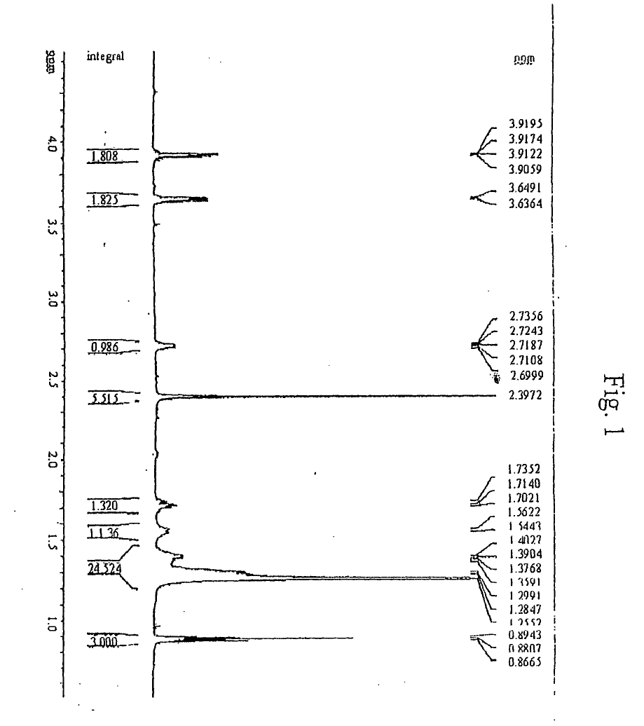

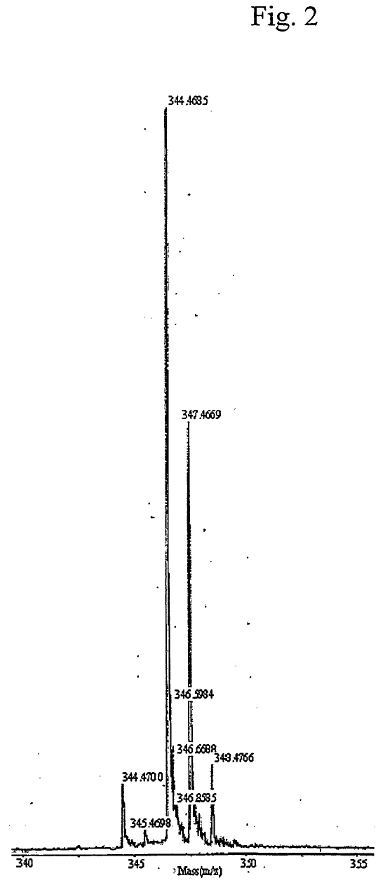

First, the present inventors prepared N,N-dimethylphytosphingosine of formula 1 as follows: 2 g (0.0063 mol) of phytosphingosine was added to 200 ml of methanol, stirred at 40° C. to dissolve it. Then, 200 ml of 0.2 M borate buffer (pH 9.0) was added slowly, and then the solution was dispersed with sonication. Subsequently, 1 g of sodium borohydride was added carefully to the dispersion in ice bath at 4° C. At this time, it should be taken care of abrupt boiling. After 10 min, 10 ml of 37% aqueous formaldehyde solution was added six times at every 5 min. After 24 hrs, sodium borohydride was added again in a same manner. A reaction was performed at room temperature for 72 hrs. After 72 hrs, 100 ml of chloroform was added, and then the reaction was terminated by extracting with distilled water. Then, the compound of formula 1 was obtained by purification with silica gel adsorption chromatography. The resulting compound was purified by silica gel thin layer chromatography (chloroform, methanol, aqueous ammonia=80:20:2, Rf=0.6), to give a white compound of formula 1 (1.5 g, yield 68.9%). ¹H NMR showed that two methyl groups were introduced (=2.4 ppm, s, 6H; FIG. 1). A molecular weight was determined with MALDI-MASS (calculated: 346.32, found: 346.46).

### Example 2

### Inhibition of Sphingosine Kinase Activity of N,N-dimethylphytosphingosine

The present inventors performed experiment as follows to demonstrate that the composition of the present invention had an inhibitory effect of sphingosine kinase.

The same experiment was performed on dimethylsphingosine to compare with the effect of dimethylphytosphingosine of the present invention. Sphingosine kinase assay buffer was prepared as follows to determine an activity of sphingosine kinase: 20 mM Tris buffer, pH 7.2, 10 mM MgCl₂, 20% glycerol, 1 mM dithiothreitol, 1 mM Na₃VO₄, 15 mM NaF, 10 g/ml leupeptin and aprotinin, 1 mM PMSF and 0.5 mM 4-deoxypyridoxine.

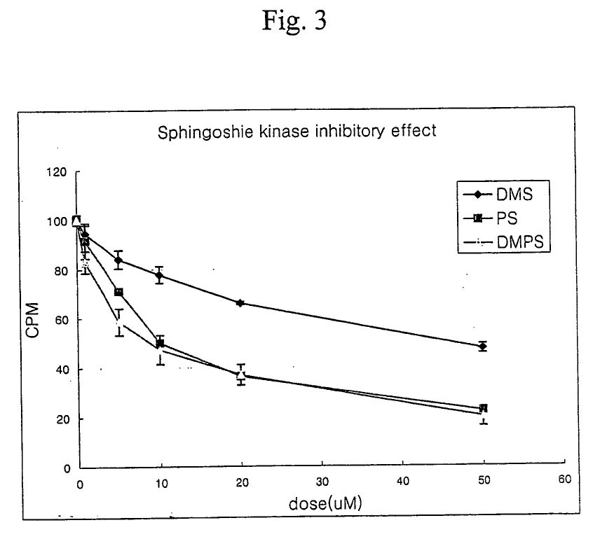

The reaction was 200 μl, each 50 μM dimethylsphingosine and dimethylphytosphingosine dissolved in 0.25% Triton X-100, 10 ng of sphingosine kinase from mouse and 1 mM [32P] ATP were added, and reacted at 37° C. for 20 min. After completion of the reaction, the reaction was terminated by adding 20˜50 μl of 1N HCl. Following termination, lipid was separated and purified by adding 1 ml of chloroform, and dried under nitrogen gas. An inhibitory effect against sphingosine kinase was found by measuring isotope with scintillation counter and then determining produced sphingosine-1-phosphate. The result was shown in FIG. 3. In the graph, indicated in FIG. 3, Y axis represents CPM and X axis represents concentration (μM). As shown in FIG. 3, dimethylphytosphingosine had more strong inhibitory effect than that of dimethylsphingosine, which suggests that it directly inhibits sphingosine kinase.

### Example 3

### Apoptosis Inducing Effect of N,N-dimethylphytosphingosine on HL60 Cell

An apoptosis inducing effect of N,N-dimethylphytosphingosine was assayed. Anti-cancer effect is expressed through various signal transduction pathway depending on working mechanism and chemical structure, but consequently give rise to apoptosis which allows cells to be killed. To demonstrate anti-cancer effect of N-N-dimethylphytosphingosine on cancer cell, first the degree of cytotoxicity was measured, and then apoptosis was found based on the results.

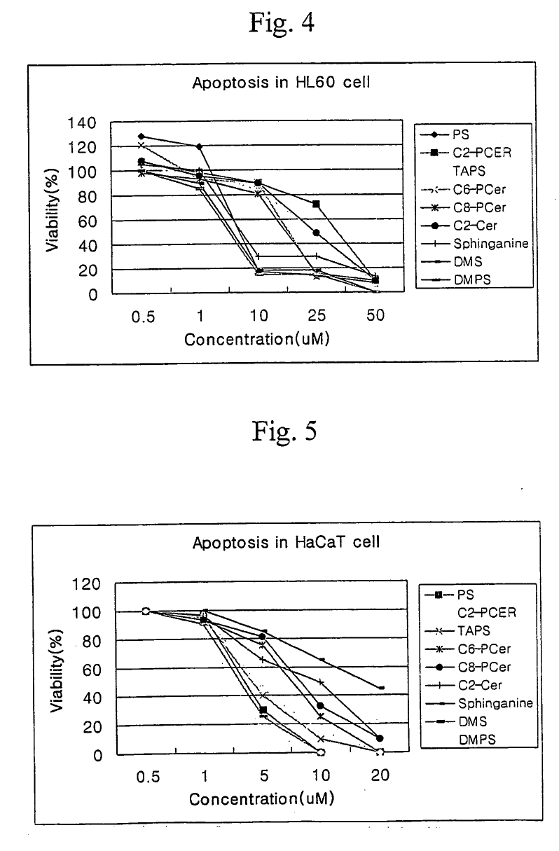

This experiment was performed by MTT assay. MTT (3-[4,5-Dimethylthiazol-2-yl]-2,5-diphenyltetrazolium) is a staining reagent displaying yellow color when it is dissolved in medium, but it is discolored to violet formazan by active dehydrogenase in mitochondria of viable cell. Accordingly, when cells do not grow or die, the discoloration to violet is reduced, and the degree of reduction is measured by absorption spectrophotometry. HL60 cell lines were seeded to 96-well plate at appropriate concentration, and incubated in 5% CO₂ incubator at 37° C. for 24 hrs. Thereafter, individual sample to be assessed on apoptosis effect, i.e. phytosphingosine, C2 phytoceramide, tetraacetylphytosphingosine, C6 phytoceramide, C8 phytoceramide, C3 ceramide, sphinganine, dimethylsphingosine and dimethylphytosphingosine was diluted with serum free RPMI. Cells were treated with 0.5 M˜50 M of the dilutions, and incubated for 24 hrs. MTT (final concentration: 0.5 mg/ml) was loaded to the individual well. After incubating for another 3 hrs and dissolving the staining reagent with pipette, the absorbance was measured at 570 nm. The results were shown in FIG. 4.

As shown in FIG. 4, N,N-dimethylphytosphingosine of the present invention induced cell death on HL60 cell, and thus it was considered to have an anti-cancer effect for inducing apoptosis.

The following is the meaning of the abbreviations used in FIG. 4.
PS: phytosphingosine,
C2-PCER: C2 phytoceramide(N-acetylphytosphingosine),
TAPS: tetraacetylphytosphingosine,
C6-PCER: C6 phytoceramide,
C8-PCer: C8 phytoceramide,
C2-Cer: C2 ceramide,
Sphiganine: Sphinganine
DMS: dimethylsphingosine
DMPS: dimethylphytosphingosine

### Example 4

### Apoptosis Inducing Effect of N,N-dimethylphytosphingosine on HaCaT Cell

This Experiment was carried out in an identical manner to that described for example 3, except for using HaCaT cell to induce apoptosis. The results were shown in FIG. 5.

As shown in FIG. 5, N,N-dimethylphytosphingosine of the present invention induced cell death on HaCaT cell, and thus it was considered to have an anti-cancer effect for inducing apoptosis. Particularly, it was shown that the effect of the N,N-dimethylphytosphingosine was far superior to that of dimethylsphingosine. The meanings of the abbreviations used in FIG. 5 are identical to that described in example 3.

### Example 5

### Apoptosis Inducing Effect of N,N-dimethylphytosphingosine on LLC-PK1 cell

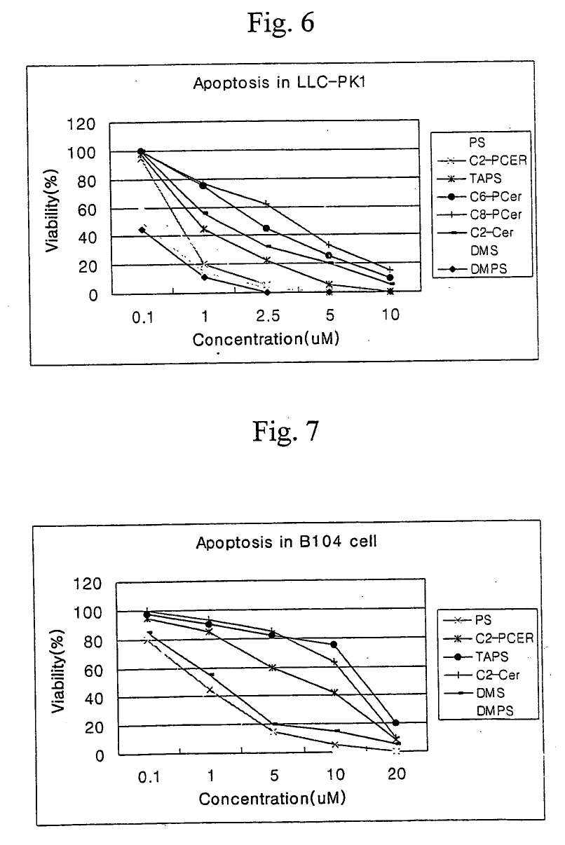

This Experiment was carried out in an identical manner to that described for example 3, except for using LLC-PK1 cell to induce apoptosis. The results were shown in FIG. 6.

As shown in FIG. 6, N,N-dimethylphytosphingosine of the present invention induced cell death on LLC-PK1 cell, and thus it was considered to have an anti-cancer effect for inducing apoptosis. Particularly, it was shown that the effect of the N,N-dimethylphytosphingosine was far superior to that of dimethylsphingosine. The meanings of the abbreviations used in FIG. 6 are identical to that described in example 3.

### Example 6

### Apoptosis Inducing Effect of N,N-dimethylphytosphingosine on B104 Cell

This Experiment was carried out in a manner identical to that described for example 3, except for using B104 cell to induce apoptosis. The results were shown in FIG. 7.

As shown in FIG. 7, N,N-dimethylphytosphingosine of the present invention induced cell death on B104 cell, and thus it was considered to have an anti-cancer effect for inducing apoptosis. Particularly, it was shown that the effect of the N,N-dimethylphytosphingosine was far superior to that of dimethylsphingosine. The meanings of the abbreviations used in FIG. 7 are identical to that described in example 3.

### Example 7

### Apoptosis Inducing Effect of N,N-dimethylphytosphingosine on MDA-MB-231 Cell

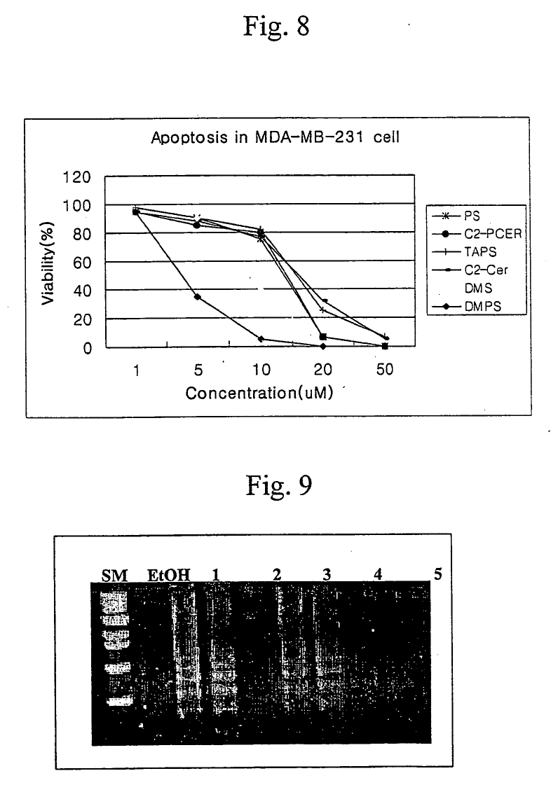

This Experiment was carried out in an identical manner to that described for example 3, except for using MDA-MB-231 cell to induce apoptosis. The results were shown in FIG. 8.

As shown in FIG. 8, N,N-dimethylphytosphingosine of the present invention induced cell death on MDA-MB-231 cell, and thus it was considered to have an anti-cancer effect for inducing apoptosis. Particularly, it was shown that the effect of the N,N-dimethylphytosphingosine was far superior to that of dimethylsphingosine. The meanings of the abbreviations used in FIG. 8 are identical to that described in example 3.

### Example 8

### DNA Fragmentation of N,N-dimethylphytosphingosine

DNA fragmentation as a representative characteristic of apoptosis was assessed at a level of concentration including cytotoxicity. Apoptosis is a programmed cell death, characterized by complicate biological characteristics including morphological changes, chromatin condensation, a formation of apoptotic body and the like. This experiment was performed to assess DNA fragmentation among them. HL60 cells were seeded (1×10⁷ cell/10 ml), and incubated in 5% CO₂ incubator at 37° C. for 24 hrs. Then, N,N-dimethylphytosphingosine and control substances (at concentration indicated in FIG. 9) was added and incubated for 24 hrs. All of substances were dissolved in EtOH. Cells were recovered by centrifugation, and then cell membrane was disrupted by adding lysis buffer (5 mM Tris-HCl (PH 7.4), 20 mM EDTA, 0.5% Triton X-100). After centrifuging at 12,000 rpm for 10 min, supernatant in which DNA fragments were dissolved was recovered. To the supernatant was added equivalent amount of phenol, vortexed, centrifuged at 12,000 rpm for 10 min, and then supernatant was recovered carefully. DNA extraction with phenol:chloroform:isoamylalcohol (25:24:1) or chloroform was performed in a same manner as the method with phenol. To the supernatant which was treated by various solvents was added a solution of 0.3 M sodium acetate (pH 5.2) in EtOH, and precipitated in a freezer at −20° C. for 24 hrs. After centrifuging at 12,000 rpm for 10 min, supernatant was decanted to obtain DNA pellets. 70% EtOH was added to the remaining DNA pellets to wash them. Again, after centrifuging, supernatant was discarded, the remaining DNA fragments were dissolved in TE buffer. To remove RNA in addition-to DNA fragments, 1 l of 0.5 mg/ml RNAse was added, and reacted at 37° C. for 30 min. DNA fragmentation was found by 1.2% agarose gel electrophoresis. The results were shown in FIG. 9. As shown in FIG. 9, N-acylsphingosine (C2-ceramide) among short-chain ceramides induced DNA fragmentation at 25.0 μM. N-acylsphingosine was known as apoptosis inducing substance, and DNA fragmentation was one example of such effects. Likewise, it was found that N,N-dimethylphytosphingosine of the present invention could induce DNA fragmentation at the same concentration (25.0 μM). It should be noted that N,N-dimethylphytosphingosine induced obvious ladder-like DNA fragmentation at a lower concentration compared to C2-ceramide used as control, and the degree of the fragmentation was stronger than C2 ceramide. Accordingly, it is considered that N,N-dimethylphytosphingosine of the present invention has excellent apoptosis inducing effect compared to N-acylsphingosine. It was not observed that CLA ceramide belonging to sphingosineceramide induced DNA fragmentation.

The following is the meaning of the symbols used in FIG. 9.
SM: DNA size marker
EtOH: Ethanol
1: DMPS 12.5 μM
2: DMPS 25.0 μM
3: DMPS 50.0 μM
4: C2 ceramide 25.0 μM
5: C2 ceramide 50.0 μM
6: CLA ceramide 25.0 μM
7: CLA ceramide 50.0 μM

### Example 9

### The Effect of N,N-dimethylphytosphingosine on Anti-Histone Antibody Binding

This experiment was based on the theory of ELISA. Briefly, Mono- or oligosome of degraded nucleic acid in cytoplasm can be detected by means of monoclonal antibodies which are specific for histones (H2A, H2B, H3 and H4) and single-stranded or double-stranded DNA. Cells which apoptosis was induced activated Ca2+ and Mg2+-dependent endonuclease, and the enzyme degraded adjacent double stranded DNA to form mono- or oligosome. On this account, histone which had been compactly bound to the DNA in nucleus was exposed to outside.

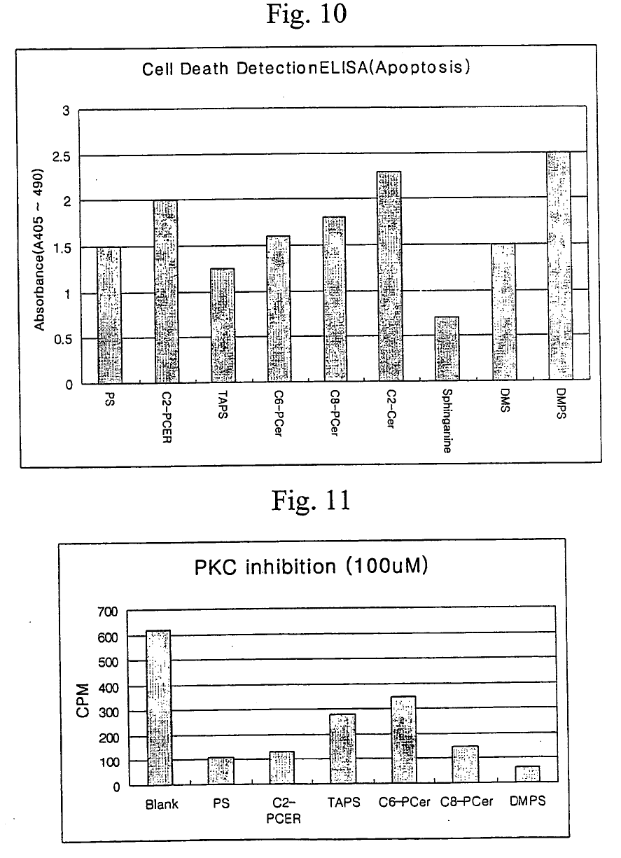

First, anti-histone antibody was immobilized to 96 well plate. Coating solution was added, and reaction was performed at ambient temperature for 1 hr. Then, each sample was treated to obtain cell lysates, and histone component presented in nucleosome of cytoplasm of cell lysates was adhered to the 96 well plate to which anti-histone antibody was immobilized. Above procedure was performed at 15˜25° C. for 90 min. Then, anti-DNA-peroxidase (POD) was bound to DNA part of nucleosome of cytoplasm. This procedure was performed at ambient temperature for 90 min. Unbound peroxidase conjugate was washed, ABTS(2,2′-azino-di-[3-ethylbenzthiazoline sulfonate]) was added as an substrate, reacted for 10˜20 min, and then absorbance was measured at 405 nm. The results were shown in FIG. 10.

As shown in FIG. 10, DMPS caused higher absorbance (A405 nm-A490 nm) than other controls. This means that mono- or oligosome have been increased much, and consequently can be understood that DNA fragmentation was induced in the sample treated with DMPS compared to other samples. Much DNA fragmentation is indicative of much apoptosis.

As shown in the results of examples 3 to 9, N,N-dimethylphytosphingosine of the present invention induced cell death of immunonological cell line, skin cancer cell line, melanocyte, lung cancer cell line and breast cancer cell line, and dimethylsphingosine of the present invention had the strongest cytotoxicity on cancer cell line compared to other sphingolipid derivatives.

### Example 10

### PCK Inhibition Effect of N,N-dimethylphytosphingosine

The present inventors assessed an inhibitory effect of N,N-dimethylphytosphingosine on protein kinase C(PKC) using epidermis cells of rat. Epidermis cells were cultured to 2×10⁷ cell/ml. N,N-dimethylphytosphingosine and other sphingolipid derivatives (100 μM and 400 μM respectively) were added, and reacted. The cells were washed with PBS and disrupted with homogenizer. The disruption of cells was centrifuged, and the supernatant was passed through DE52 column to obtain a portion containing protein kinase C. For activated PKC reaction, a tube containing 5 μl of PKC coactivation 5× buffer, PKC activation 5× buffer, PKC biotinylated peptide substrate, [32P]ATP mix respectively were prepared as a control. Also, other individual tubes containing 5 μl of PKC coactivation 5× buffer, control 5× buffer, PKC biotinylated peptide substrate, [32P]ATP mix were prepared further containing 5 μl of the relevant enzyme in each tube. The reaction was performed at 30° C. for 5 min. Thereafter, terminating solution (12.5 μl) was added to quench the reaction. The reaction solution (10 μl) was dropped on SAM2™ membrane, washed with 2 M NaCl (1×, 30 seconds), 2 M NaCl (3×, 2 min), 1% H₃PO₄ and 2M NaCl solution (4×, 2 min), and distilled water (2×, 30 seconds), dried and then measured isotope to assess the effect of PKC inhibition. The results were in FIG. 11. As shown in FIG. 11, N,N-dimethylphytosphingosine has stronger inhibitory effect than any other compound on PKC. Thus, the composition of the present invention was considered to have an anti-inflammatory.

### Example 11

### Antibacterial Effect of N,N-dimethylphytosphingosine

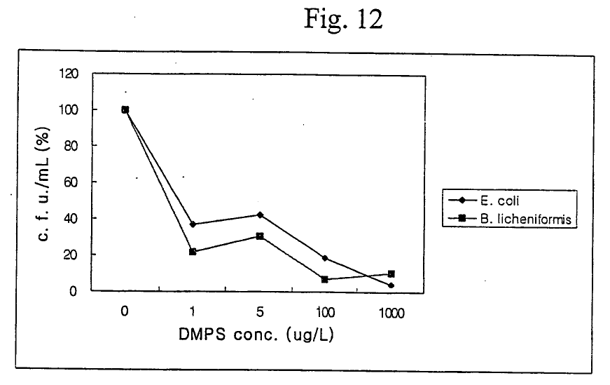

The present inventors performed experiment using Bacillus licheniformis (Gram positive bacteria) and E. coli(Gram negative bacteria) to test an antibacterial activity of N,N-dimethylphytosphingosine. In this experiment, autoclaved LB (bactopeptone 10 g/l, yeast extracts 5 g/l, and sodium chloride 10 g/l) or TS (triptone 15 g/l, soitone 5 g/l, and sodium chloride 5 g/l) was used as culture medium, and cultured at 30° C. or 37° C. for 2-3 days. After culturing, an antibacterial capacity was measured by counting the number of cells. N,N-dimethylphytosphingosine was used in the form of solution in EtOH, and used successively at 1 μg/ml, 5 μg/ml, 100 μg/ml, 1,000 μg/ml to affirm an antibacterial activity. Each microbe was cultured, diluted successively with 10-fold, smeared on the medium, cultured again, and then dilution rate which formed 30-300 populations per plate medium was determined. After culturing each microbe, the culture was diluted with the determined dilution rate. At this time, 0.85% NaCl was used as a diluted solution. A sample which was prepared by above mentioned method was diluted successively with a solvent for preparing the sample. After obtaining the desired concentration, 1 ml of the diluted sample was added to 9 ml of a diluted solution of microbes, and then mixed thoroughly. After standing at 30° C. or 37° C. for 1 hr (with mixing occasionally), each 100 μl was smeared on the culture medium. After culturing under individual condition, the number of population was measured. The results were shown in FIG. 12.

As shown in FIG. 12, a number of colonies reduced in both E. coli and B. licheniformis, and a small amount of 1 μg/l could reduced a amount of colonies to 40%.

The invention will now be illustrated by the following non-limiting examples of the formulation type:

### Formulation Example 1

### Cream Containing 2% N,N-dimethylphytosphingosine

TABLE 1

| Components | wt % |
|---|---|
| N,N-dimethylsphingosine | 2.0% |
| Propylene glycol | 20.0% |
| Stearyl alcohol | 6.5% |
| Cetyl alcohol | 3.5% |
| Sorbitan monostearate | 3.0% |
| Polysorbate 60 | 2.0% |
| Isopropyl myristate | 1.0% |
| Anhydrous sodium sulphite | 0.2% |
| Polysorbate 80 | 0.1% |
| Purified water | 61.7% |

Stearyl alcohol, cetyl alcohol, sorbitan monostearate and isopropyl myristate were introduced to double wall container, and the mixture was heated to be completely dissolved. The mixture was homogenized with homogenizer for liquid at 70 to 75° C., then added to the mixture of separate purified water, propylene glycol and polysorbate 60. The resulting emulsion was cooled to less than 25° C. with continuous mixing. A solution of N,N-dimethylphytosphingosine, polysorbate 80 and purified water, and a solution of anhydrous sodium sulphite in purified water were added to the emulsion with continuous mixing. After homogenizing a cream, a suitable tube was charged with it.

### Formulation Example 2

### Topical Gel Containing 2% N,N-dimethylphytosphingosine

TABLE 2

| Components | wt % |
|---|---|
| N,N-dimethylsphingosine | 2.0% |
| Propylene glycol | 4.0% |
| Hydroxypropyl beta-cyclodextrin | 25.0% |
| EtOH 95% (v/v) | 4.0% |
| Carrageenan PJ | 1.0% |
| Purified water | To 100% |

An appropriate amount of hydrochloric acid was added to the mixture to give a solution. An appropriate amount of sodium hydroxide was added to the solution to adjust the pH of the solution to 6.0. An appropriate amount of purified water was added to the solution to give a 100 mg of the solution.

To a solution of hydroxypropyl beta-cyclodextrin in purified water was added N,N-dimethylphytosphingosine with stirring. An appropriate amount of hydrochloric acid was added to the mixture to give a solution. An appropriate amount of sodium hydroxide was added to the solution to adjust the pH of the solution to 6.0. To this solution was added a dispersion of carageenan PJ in propylene glycol with mixing. The mixture was heated to 50° C. with slowly mixing. EtOH was added to the mixture, and then cooled to about 35° C. The remaining purified water was added, and then mixed to give a homogeneous mixture.

### Formulation Example 3

### Topical Cream Containing 2% N,N-dimethylphytosphingosine

TABLE 3

| Components | wt % |
|---|---|
| N,N-dimethylphytosphingosine | 2.0% |
| Hydroxypropyl beta-cyclodextrin | 20.0% |
| Stearyl alcohol | 2.5% |
| cetyl alcohol | 2.5% |
| Mineral oil | 11.0% |
| glycerol monostearate | 2.5% |
| Glycerol | 5.0% |
| sorbate 60 | 2.0% |
| Polysorbate 60 | 3.5% |
| Purified water | To 100% |

An appropriate amount of hydrochloric acid was added to the mixture to give a solution. An appropriate amount of sodium hydroxide was added to the solution to adjust the pH of the solution to 6.0. An appropriate amount of purified water was added to the solution to give a 100 mg of the solution.

To a solution of hydroxypropyl beta-cyclodextrin in purified water was added N,N-dimethylphytosphingosine with stirring. An appropriate amount of hydrochloric acid was added to the mixture to give a solution. An appropriate amount of sodium hydroxide was added to the solution to adjust the pH of the solution to 6.0.

To the mixture was added glycerol and polysorbate 60 with stirring and the mixture was heated to 70° C. The resulting mixture was added to a mixture of mineral oil, stearyl alcohol, cetyl alcohol, stearyl monostearate and sorbate 60 with slowly mixing at 70° C. After cooling less than 25° C., The remaining purified water was added, and then mixed to give a homogeneous mixture.

### Formulation Example 4

### Liposome Formulation Containing 2% N,N-dimethylphytosphingosine

TABLE 4

| Components | wt % |
|---|---|
| N,N-dimethylphytosphingosine | 2.0% |
| Phosphatidylcholine | 30.0% |
| Cholesterol | 5.0% |
| EtOH | 10.0% |
| Methyl paraffin | 0.15% |
| Propyl paraffin | 0.02% |
| Disodium edetate | 0.15% |
| NaCl | 0.4% |
| Hydroxypropyl methylcellulose | 1.2% |
| Purified water | to 100% |

A mixture of N,N-dimethylphytosphingosine, phosphatidylcholine, cholesterol and EtOH was stirred and heated at 55 to 60° C. to give a solution. To the mixture was added a solution of methyl paraffin, propyl paraffin, disodium edetate and sodium chloride in purified water with homogenizing. hydroxypropyl methylcellulose in purified water was added, and then mixed continuously until swelling.

### Formulation Example 5

### Liposome Formulation Containing 2% N,N-dimethylphytosphingosine

TABLE 5

| Components | wt % |
|---|---|
| N,N-dimethylphytosphingosine | 2.0% |
| Phosphatidylcholine | 10.0% |
| Cholesterol | 1.0% |
| EtOH | 7.5% |
| Hydroxypropyl methylcellulose | 1.5% |
| Sodium hydroxide(1 N) | to adjust pH 5.0 |
| Purified water | to 100% |

A mixture of phosphatidylcholine and cholesterol in EtOH was stirred and heated at 40° C. to give a solution. N,N-dimethylphytosphingosine was dissolved in purified water with mixing at 40° C. To the aqueous solution was added slowly alcoholic solution with homogenizing over 10 min. Hydroxypropyl methylcellulose in purified water was added, and then mixed continuously until swelling. The resulting solution was adjusted to pH 5.0 by adding 1 N sodium hydroxide and diluted with the remaining purified water.

### Formulation Example 6

### Nanodispersion of N,N-dimethylphytosphingosine

TABLE 6
N,N-dimethylphytosphingosine nanodispersion(phase inversion)

| Components | wt % |
|---|---|
| N,N-dimethylphytosphingosine | 36.6% |
| Phosphatidylcholine | 9.0% |
| Polysorbate 80 | 34.0% |
| EtOH | 7.4% |
| Myglyol 812 | 13.0% |

Myglyol 812, N,N-dimethylphytosphingosine and polysorbate 80 were mixed. Adding phosphatidylcholine dissolved in EtOH to the mixture to give homogeneous clear liquid.

TABLE 7
Nanodispersion of N,N-dimethylphytosphingosine(aqueous phase)

| Components | wt % |
|---|---|
| N,N-dimethylphytosphingosine | 2.0% |
| Phosphatidylcholine | 0.49% |
| Polysorbate 80 | 1.86% |
| EtOH | 0.63% |
| Myglyol 812 | 0.71% |
| Purified water | to 100.0% |

Aqueous phase containing N,N-dimethylphytosphingosine (for example, 94.54 g) was placed in a container with stirring at 50° C. The liquid nanodispersion phase inversion (for example, 5.46 g) was added to the aqueous phase with stirring.

### Formulation Example 7

### Medical Ointment Base Formulations

TABLE 8

| Components | wt % |
|---|---|
| Lanolin Alcohol | 1 |
| stearyl alcohol | 2 |
| Ceteareth-20 | 2 |
| Perlatum | 84.5 |
| Lecithin | 1.5 |
| Caprylic/Capric Triglyceride | 2 |
| PEG20 Corn Glycerides | 5 |
| DMPS | 2 |

### Formulation Example 8

### Cosmetic Cream Formulations

TABLE 9

| | INCI Name | wt % |
|---|---|---|
| Aqueous | Disodium EDTA | 0.020 |
| phase | Glycerine | 4.000 |
| | Buthylene glycol | 2.000 |
| | Xanthan gum | 0.030 |
| | Triethanolamine | 0.200 |
| | Di-water | to 100 |
| | Carbomer | 0.1 |
| Oil | Stearic Acid | 1.800 |
| phase | Glyceryl Stearate PEG-100 Stearate | 1.000 |
| | Stearyl alcohol | 2.000 |
| | Glyceryl stearate | 2.000 |
| | Sorbitan sesquioleate | 0.300 |
| | Polysorbate 60 | 1.200 |
| | Mineral oil | 6.000 |
| | Isopropyl myristate | 1.500 |
| | Cetyl octanoate | 1.000 |
| | DMPS | 2.000 |
| | Dimethicone | 0.400 |
| | Preservative | Q.S |

Aqueous phase and oil phase were heated to 75° C. respectively.

After checking complete dissolution of the aqueous phase and the oil phase, the aqueous phase was introduced to a major oven.

The aqueous phase in the major oven was stirred using homomixer (3,500 rpm) and peddlemixer (30 rpm) for 3 min, and then was cooled.

According to the present invention as described above, various mechanisms which sphingosine kinase causes can be blocked by using the composition of the present invention that can inhibit the activity of sphingosine kinase. For example, the composition can maintain a high level of ceramide and sphingosine by blocking the phosphorylation of ceramide and sphingosine with sphingosine kinase, such a high level of ceramide and sphingosine can induce apoptosis on cancer cell. Accordingly, cancer can be treated or prevented. Also, hyperplastic diseases, for example cancer and psoriasis, etc. can be treated or prevented by inhibiting the activity of sphingosine kinase which promotes cell proliferation. Also, various mechanisms which protrin kinase C causes can be blocked by using the composition of the present invention that can inhibit the activity of protrin kinase C. Particularly, since the composition has a excellent effect which inhibits an inflammation, when it is administrated together with other anti-cancer drug, excellent anti-cancer effect can be expected. Also, the composition of the present invention can be used exclusively in cases which require apoptosis, since the composition has an excellent apoptosis inducing activity as such. Further, the composition of the present invention has an excellent antibacterial activity.

Although preferred embodiments of the present invention have been described for illustrative purposes, those skilled in the art will appreciate that various modifications, additions and substitutions are possible, without departing from the scope and spirit of the invention as disclosed in the accompanying claims.

1. An anti-cancer composition comprising N,N-dimethylphytosphingosine as an active ingredient.

2. The composition according to claim 1, further comprising at least one phytosphingosine derivatives selected from the group consisting of phytosphingosine, acetylated phytosphingosine and ethylated phytosphingosine.

3. The composition according to claim 2, wherein the weight ratio of N,N-dimethylphytosphingosine to phytosphingosine derivatives is 1:1.

4. A kit for treating cancer comprising the composition of claim 1.

5. The kit of claim 4, comprising the composition as an adjuvants of other anti-cancer drugs.

6. A sphingosine kinase inhibitor composition comprising N,N-dimethylphytosphingosine as an active ingredient.

7. An apoptosis inducing composition comprising N,N-dimethylphytosphingosine as an active ingredient.

8. A protein kinase inhibitor composition comprising N,N-dimethylphytosphingosine as an active ingredient.

9. (canceled)

10. A composition for treating a hyperplastic disease comprising N,N-dimethylphytosphingosine as an active ingredient.

11. The composition according to claim 10, wherein the hyperplastic disease is psoriasis.

12. (canceled)

13. (canceled)
